(19) United States
(12) Patent Application Publication
(10) Pub. No.: US 2001/0046514 A1
(43) Pub. Date: Nov. 29, 2001

(54) Medicated sock

(76) Inventor: Alex Loaiza, Canyon Country, CA (US)

Correspondence Address: Matthew J. Peirce, Esq., 1550 Starlight Canyon Avenue, Las Vagas, NV, 89123, US

(21) Appl. No.: 09/863,241

(22) Filed: May 22, 2001

Related U.S. Application Data
(60) Provisional application No. 60/206,310, filed on May 23, 2000.

(51) Int. Cl. A61K 9/70; A61F 13/00

(52) U.S. Cl. 424/443; 442/123

(57) ABSTRACT

A new and improved medical device is disclosed. The present invention would be a medicated sock or pair of socks which would be worn under a normal pair of socks. The present invention would have anti-fungal medicine incorporated into the fabric of the socks or socks used with the present invention, which would be used to fight athlete's foot or any other type of foot ailment which would be treated with medicine. The present invention reduces or eliminates the time and effort needed when applying medicine directly to the foot of a user.

### I. BACKGROUND OF THE INVENTION

The present invention concerns that of a new and improved medical device, and more particular relates to a pair of socks which have incorporated medicine in their fabric.

### II. DESCRIPTION OF THE PRIOR ART

U.S. Pat. No. 5,807,890, issued to Yu et al., discloses a therapeutic treatment as well as prophylactic measures that are provided by topical application to eradicate or prevent the development of axillary foul odor, foot malodor and other body odors.

U.S. Pat. No. 4,865,844, issued to Blank et al., discloses a therapeutic method of treating the chronic human superficial fungus infection tinea pedis.

### III. SUMMARY OF THE INVENTION

The present invention concerns that of a new and improved medical device. The present invention would be a medicated sock or pair of socks which would be worn under a normal pair of socks. The present invention would have anti-fungal medicine incorporated into the fabric of the socks or socks used with the present invention, which would be used to fight athlete's foot or any other type of foot ailment which would be treated with medicine. The present invention reduces or eliminates the time and effort needed when applying medicine directly to the foot of a user.

There has thus been outlined, rather broadly, the more important features of a medical device in order that the detailed description thereof that follows may be better understood and in order that the present contribution to the art may be better appreciated. There are, of course, additional features of the medical device that will be described hereinafter and which will form the subject matter of the claims appended hereto.

In this respect, before explaining at least one embodiment of the medical device in detail, it is to be understood that the medical device is not limited in its application to the details of construction and to the arrangements of the components set forth in the following description or illustrated in the drawings. The medical device is capable of other embodiments and being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present medical device. It is important, therefore, that the claims be regard as including such equivalent constructions insofar as they do not depart from the spirit and scope of the present invention.

It is therefore an object of the present invention to provide a medical device which has all of the advantages of the prior art and none of the disadvantages.

It is another object of the present invention to provide a medical device which may be easily and efficiently manufactured and marketed.

It is another object of the present invention to provide a medical device which is of durable and reliable construction.

It is yet another object of the present invention to provide a medical device which is economically affordable and available for relevant purchasing government entities.

Other objects, features and advantages of the present invention will become more readily apparent from the following detailed description of the preferred embodiment when considered with the attached drawings and appended claims.

### IV. BRIEF DESCRIPTION OF THE DRAWINGS

FIG. 1 is a side view of the present invention.

FIG. 2 shows the present invention in use, under a pair of normal socks.

### V. DESCRIPTION OF THE PREFERRED EMBODIMENT

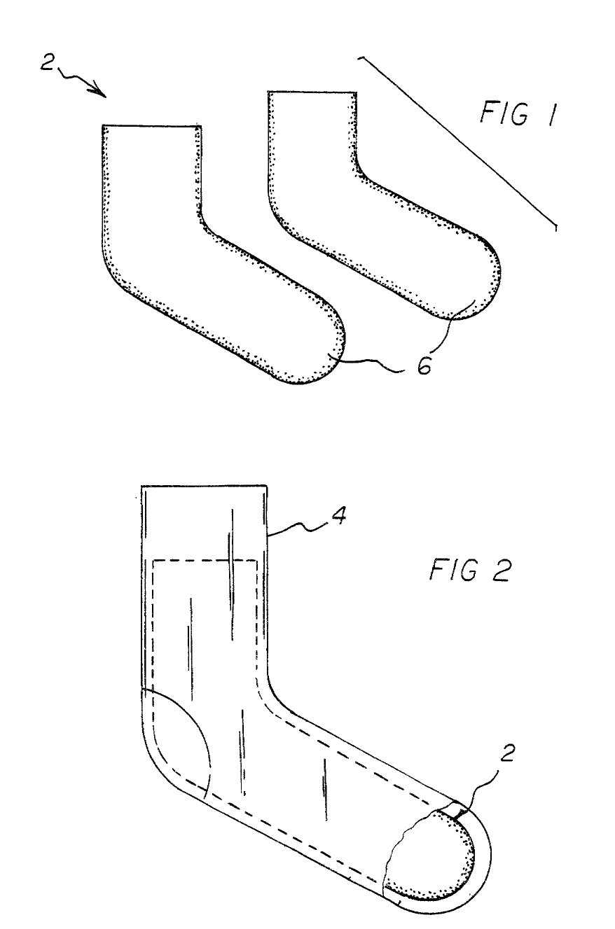

FIG. 1 is a side view of the present invention. The present invention concerns that of a new and improved medical device. The present invention would be a medicated sock 2 or pair of socks 2 which would be worn under a normal pair of socks 4. The present invention would have anti-fungal medicine 6 incorporated into the fabric of the socks or socks used with the present invention, which would be used to fight athlete's foot or any other type of foot ailment which would be treated with medicine. The present invention reduces or eliminates the time and effort needed when applying medicine directly to the foot of a user.

FIG. 2 shows the present invention in use, under a pair of normal socks 4. The present invention could be used with either one foot or both feet, depending on the specific need of a user. The present invention, whether used with one foot or two feet, could be worn with a normal pair of socks 4 of any height. The present invention would be made available in a wide variety of sizes to accommodate people with different feet sizes. The present invention would preferably be made of a thin nylon-type material that would hug a person's feet and prevent the present invention from slipping off while in use. After use, the present invention would be disposable.

The prior art does not teach or show the use of a sock or pair of socks that has medicine incorporated into the very fabric of the sock or pair of socks. The prior art also does not teach the method of applying a sock or pair of socks that has medicine incorporated into the very fabric of the sock or pair of socks to human skin and maintaining that contact over a long period of time. Further, the prior art does not teach or show the use of a sock or pair of socks that has medicine incorporated into the very fabric of the sock or pair of socks that is disposable.

What I claim as my invention is:

1. A medical device comprising:
(a) a first medical sock fabricated from a nylon-type material, the sock having medicine incorporated into the fabric of the sock, and
(b) a second medical sock fabricated from a nylon-type material, the sock having medicine incorporated into the fabric of the sock,
(c) wherein a user would wear the first medical sock on one foot and the second medical sock on the other foot.

2. A medical device according to claim 1 wherein the medicine incorporated into the fabric of the first medical sock and the medicine incorporated into the fabric of the second medical sock would be anti-fungal medicine.

3. A medical device according to claim 2 wherein the first medical sock and the second medical sock are disposable after one-time use.

4. A medical device according to claim 3 in combination with a pair of regular socks, the pair of regular socks comprising a first sock and a second sock, the first sock designed to be placed over the first medical sock after the first medical sock had been put on a person's foot, and the second sock designed to be placed over the second medical sock after the second medical sock had been put on a person's other foot.

5. A method of applying medicine to a person's feet wherein the medicine is incorporated into a medical device which maintains constant and continual contact with the person's feet, comprising the steps of:
(a) applying a first medical sock onto a user's foot, the first medical sock being fabricated from a nylon-type material, the sock having medicine incorporated into the fabric of the sock,
(b) applying a second medical sock onto a user's other foot, the second medical sock being fabricated from a nylon-type material, the sock having medicine incorporated into the fabric of the sock,
(c) applying a first regular sock onto a user's foot over the first medical sock after the first medical sock had been placed onto a user's foot, the first regular sock completely covering the first medical sock,
(d) applying a second regular sock onto a user's foot over the second medical sock after the second medical sock had been placed onto a user's foot, the second regular sock completely covering the second medical sock, and
(e) maintaining the first medical sock and the second medical sock on a user's feet for a length of time.
